(19) United States
(12) Patent Application Publication
(10) Pub. No.: US 2023/0133899 A1
(43) Pub. Date: May 4, 2023

(54) COSMETIC COMPOSITION FOR ANTIOXIDANT OR SKIN INFLAMMATION IMPROVEMENT CONTAINING EUPHORBIA SUPINA EXTRACT

(71) Applicant: ChungdamCDCJN Pharm LLC., Chuncheon-si (KR)

(72) Inventors: Sung Pil KWON, Seoul (KR); Chang soon YOON, Chuncheon-si (KR); Ji An KIM, Chuncheon-si (KR); Ji Won KIM, Chuncheon-si (KR); NaYaeon JANG, Chuncheon-si (KR); Dongcheol JANG, Chuncheon-si (KR)

(21) Appl. No.: 17/536,877

(22) Filed: Nov. 29, 2021

(30) Foreign Application Priority Data
Oct. 29, 2021 (KR) 10-2021-0146868

(51) Int. Cl. A61K 8/9789; A61Q 19/08; B01D 11/02

(52) U.S. Cl. CPC A61K 8/9789; A61Q 19/08; B01D 11/0288; B01D 11/0292

(57) ABSTRACT

The present invention relates to a cosmetic composition for antioxidant or skin inflammation improvement containing a Euphorbia supina extract, and it is possible to efficiently extract a Euphorbia supina extract to provide a cosmetic composition which exhibits an excellent antioxidant or skin inflammation improvement effect with a high content of ellagic acid. In addition, by applying the same, it is possible to provide a cosmetic composition in a stable multilamellar liposome formulation.

### CROSS-REFERENCE TO RELATED APPLICATION

This application claims priority to and the benefit of Korean Patent Application No. 10-2021-0146868, filed on Oct. 29, 2021, the disclosure of which is incorporated herein by reference in its entirety.

### TECHNICAL FIELD

The present invention relates to a cosmetic composition for antioxidant or skin inflammation improvement containing a Euphorbia supina extract, and more specifically to a cosmetic composition for antioxidant or skin inflammation improvement containing a Euphorbia supina extract with a high content of effective marker compounds.

### BACKGROUND ART

The skin is a part of the body that is directly exposed to the external environment, and it not only acts as a protective membrane to protect important organs of our body, but also controls water evaporation and protects the body from external infections. Recently, as people's interest in visually visible skin increases, the development of materials capable of enhancing various skin functions is being actively conducted.

Meanwhile, recently, fine dust among air pollutants destroys the epidermal barrier function and causes various sensitive skin symptoms such as dry skin, reduced skin regeneration rate, reduced elasticity, increased sebum secretion and inflammation and the like. In particular, it is known that keratinocytes are killed by diesel particulate extracts, which are major causative agents of fine dust, and accordingly, it is necessary to suppress the same.

Accordingly, in the present invention, it was attempted to develop raw materials of natural materials with excellent antioxidant efficacy, and to develop a cosmetic composition that can enhance the functionality for the skin which reacts sensitively to environmental changes by using a new natural product.

### RELATED ART DOCUMENTS

### Patent Documents

(Patent Document 0001) Korean Registered Patent No. 10-1671852 (Registration Date: Oct. 27, 2016) relates to a composition for skin whitening, including an extract or fraction of Euphorbia maculata L. or Euphorbia supina as an active ingredient, and it describes a composition for skin whitening, which has a skin whitening effect by inhibiting tyrosinase activity and inhibiting melanin production in melanin-producing cells.

### DISCLOSURE

### Technical Problem

The present invention is directed to providing a cosmetic composition for excellent antioxidant or skin inflammation improvement, by using a Euphorbia supina extract and selecting conditions that can exert its maximum efficacy.

### Technical Solution

The present invention provides a cosmetic composition for antioxidation, including a Euphorbia supina extract.

Meanwhile, in the cosmetic composition for antioxidation of the present invention, the Euphorbia supina extract is preferably extracted with 60% to 80% ethanol or hot water.

Meanwhile, in the cosmetic composition for antioxidation of the present invention, the Euphorbia supina extract may be collected in an aqueous layer of multilamellar liposome.

In addition, the present invention provides a cosmetic composition for improving skin inflammation, including a Euphorbia supina extract.

Meanwhile, in the cosmetic composition for improving skin inflammation of the present invention, the Euphorbia supina extract is preferably extracted with 60% to 80% ethanol or hot water.

Meanwhile, in the cosmetic composition for improving skin inflammation of the present invention, the Euphorbia supina extract may be collected in an aqueous layer of multilamellar liposome.

### Advantageous Effects

The present invention can provide a cosmetic composition that exhibits an excellent antioxidant or skin inflammation improvement effect while having a high content of ellagic acid by efficiently extracting a Euphorbia supina extract. In addition, by applying the same, the present invention can provide a cosmetic composition in a stable multilamellar liposome formulation.

### DESCRIPTION OF DRAWINGS

FIG. 1 is a graph of the results of the DPPH scavenging activity of Euphorbia supina extracts (hot water, ethanol).

FIG. 2 is a graph of the results of the total polyphenol content of Euphorbia supina extracts (hot water, ethanol).

FIG. 3 is a graph of the results of the total flavonoid content of Euphorbia supina extracts (hot water, ethanol).

FIG. 4 show chemical formulas for three types of the marker compounds of Euphorbia supina extracts.

FIG. 5 is a graph of the results of the ellagic acid content of Euphorbia supina extracts (hot water, ethanol).

FIG. 6 is a graph of the results of the DPPH scavenging activity of a Euphorbia supina hot water extract.

FIG. 7 is a graph of the results of the total polyphenol and total flavonoid contents of a Euphorbia supina hot water extract.

FIG. 8A and FIG. 8B are graphs of the results of HPLC analysis of a Euphorbia supina hot water extract for each temperature and time.

FIG. 9 is a graph of the results of the ellagic acid content of a Euphorbia supina hot water extract for each temperature and time.

FIG. 10 is a result showing the DPPH scavenging activity compared to changes in the ellagic acid content of a Euphorbia supina hot water extract.

FIG. 11 is a result showing the DPPH scavenging activity of a standard product of ellagic acid by each concentration.

FIG. 12 is a graph of the results of comparing the ellagic acid HPLC chromatograms of Euphorbia supina extracts and the control group.

FIG. 13 is a result showing the linearity of ellagic acid in a Euphorbia supina hot water extract.

FIG. 14 is a graph of the results of a multilamellar formulation including a Euphorbia supina extract.

FIG. 15 is a graph of the results showing the skin permeability of the multilamellar formulation including a Euphorbia supina extract.

FIG. 16 is a graph of the results of the stability experiments of the multilamellar formulation including a Euphorbia supina extract.

FIG. 17A and FIG. 17B are graphs of the results of the stability HPLC analysis of raw materials in the multilamellar formulation including a Euphorbia supina extract.

### MODES OF THE INVENTION

The present invention provides a cosmetic composition for antioxidation, including a Euphorbia supina extract. In addition, the present invention provides a cosmetic composition for improving skin inflammation, including a Euphorbia supina extract.

Euphorbia supina is an annual dicotyledonous plant belonging to the order Geraniales and the family Euphorbiaceae, and the stem is branched and spreads over the ground, the length is 10 to 25 cm long, and it has some hairs along with the leaves and reddish-brown spots in the center. The grass contains lactic acid, and it contains gallic acid, tannins, resins and the like in a white liquid. Euphorbia supina, which is also called milk purslane or Euphorbia humifusa Wild, is widely distributed in temperate and tropical regions such as Korea, China, Japan, Southeast Asia, North and South America and the like, and it promotes blood circulation and hemostasis, and acts to promote milk secretion and the like.

Meanwhile, the extraction method of the present invention is not limited as long as it is an extraction method known in the art, and it may be prepared using a conventional solvent under conditions of conventional temperature and pressure. For example, it may be extracted by using (a) a solvent extraction method using a solvent selected from the group consisting of water, anhydrous or hydrous lower alcohol having 1 to 4 carbon atoms (e.g., methanol, ethanol, propanol and butanol), propylene glycol, 1,3-butylene glycol, glycerin, acetone, diethyl ether, ethyl acetate, butyl acetate, dichloromethane, chloroform, hexane and a mixture thereof, (b) a supercritical extraction method by decompression by carbon dioxide and high temperature or (c) an ultrasonic extraction method. In the present invention, it is preferable to extract using 60 to 80% ethanol or hot water (80 to 100° C.), and more preferably, extraction using 60 to 80% ethanol or hot water (80 to 100° C.), which is 10 times the weight of dry matters, is good because the extraction yield may be increased. In addition, hot water extraction was used as an optimal extraction solvent than ethanol extraction because it may exhibit the efficacies of the DPPH scavenging activity, increases in the total polyphenol and flavonoid contents and a high ellagic acid content.

Meanwhile, in the cosmetic composition of the present invention, the Euphorbia supina extract may be collected in an aqueous layer of multilamellar liposome.

Liposome is an endoplasmic reticulum with a lipid bilayer structure formed using phospholipids as the main component, which are a component of biological cell membranes of amphiphilic lipids which have both a hydrophilic part that attracts water and a hydrophobic part that repels water at the same time. Multilamellar liposomes may be prepared by a known method, and after obtaining a Euphorbia supina extract by dissolving in propanediol, the multilamellar liposome formulation is formed by applying the Bangham method. The preparation process is based on the Bangham method and may be preferably prepared by the following method by modifying the same.
Step 1: Phospholipids were dissolved in propanediol. In this case, the phospholipids were preferably lecithin extracted from soybean, and it is preferable to use a phosphatidyl choline component at 95% or more, and it is preferable to use cholesterol together, but use at less than 1% and use such that the concentration of phospholipids is 5% or less.
Step 2: A lipid membrane was formed by drying with a vacuum concentrator.
Step 3: After making a Euphorbia supina extract (hot water or ethanol) to a solid content of 2% on the lipid membrane, it was hydrated with an aqueous solution.
Step 4: For homogenization of the hydrated liposome solution, a homogenizing or ultrasonic homogenizer was used.

The multilamellar liposome formulation of the present invention developed through the above process has excellent stability, and by loading useful substances in the liposome, the stability of the useful substances may be improved, and the skin permeability may be improved.

Meanwhile, for example, the cosmetic composition of the present invention may be any one selected from any one of basic cosmetic formulations selected from a solution, a suspension, an emulsion, a paste, a lotion, a gel, a water-soluble liquid, cream, essence, a surfactant-containing cleansing, oil, an oil-in-water (O/W) type and a water-in-oil (W/O) type; skin; lotion; eye cream; a soothing gel; an ointment; a formulation for mask packs; a formulation for body wash; a peeling gel; oil-in-water and water-in-oil makeup bases; a foundation; a skin cover; any one of color cosmetic formulations selected from lipstick, lip gloss, face powder, two-way cake, eye shadow, cheek color and eyebrow pencil; and a formulation for the scalp.

In addition, the cosmetic composition of the present invention may contain auxiliary agents such as hydrophilic or lipophilic active agents, preservatives, antioxidants, solvents, fragrances, fillers, blockers, pigments, odorants, dyes and the like, which are conventionally used in the art to which the present invention pertains. The amount of these various auxiliary agents is an amount conventionally used in the art, for example, 0.001 to 30 wt. % based on the total weight of the composition.

However, in any case, the auxiliary agents and the ratio thereof will be selected so as not to adversely affect the desirable properties of the cosmetic composition according to the present invention.

Meanwhile, in the cosmetic composition of the present invention, when the formulation is a solution or emulsion, a solvent, a solubilizer or an emulsifier may be used as a carrier component, and for example, water, ethanol, isopropanol, ethyl carbonate, ethyl acetate, benzyl alcohol, benzoate, propylene glycol, 1,3-butylene glycol oil, glycerol aliphatic ester, polyethylene glycol or a fatty acid ester of sorbitan may be used.

In addition, in the cosmetic composition of the present invention, when the formulation is a suspension, liquid diluents such as water, ethanol or propylene glycol, suspending agents such as ethoxylated isostearyl alcohol, polyoxyethylene sorbitol esters and polyoxyethylene sorbitan esters, microcrystalline cellulose, aluminum metahydroxide, bentonite, agar, tragacanth or the like may be used as a carrier component.

In addition, in the cosmetic composition of the present invention, when the formulation is a paste, cream or gel, animal oil, vegetable oil, wax, paraffin, starch, tragacanth, cellulose derivatives, polyethylene glycol, silicone, bentonite, silica, talc, zinc oxide or the like may be used as a carrier component.

In addition, in the cosmetic composition of the present invention, when the formulation is a powder or spray, lactose, talc, silica, aluminum hydroxide, calcium silicate or polyamide powder may be used as a carrier component, and particularly in the case of a spray, it may additionally include propellants such as chlorofluorohydrocarbons, propane/butane or dimethyl ether.

In addition, in the cosmetic composition of the present invention, when the formulation is a surfactant-containing cleanser, aliphatic alcohol sulfate, aliphatic alcohol ether sulfate, sulfosuccinic acid monoester, isethionate, imidazolinium derivatives, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester or the like may be used as a carrier component.

In addition, the cosmetic composition of the present invention may be used along with other cosmetic compositions other than the present invention. In addition, the cosmetic composition according to the present invention may be used according to a conventional method of use, and the number of times of use may be varied according to the skin condition or preference of the user.

Meanwhile, according to the following experiment, the Euphorbia supina extract of the present invention has excellent DPPH reducing capacity, the total polyphenol and flavonoid contents are increased, and the content of ellagic acid, which has excellent antioxidant, anti-inflammatory (improving skin inflammation) and cosmetic effects, is high, thereby exhibiting excellent antioxidant and skin inflammation improvement effects. In particular, it was found that the Euphorbia supina hot water extract was more excellent than the ethanol extract in the aforementioned effects and thus optimal. In addition, it was possible to prepare a multilamellar formulation to which the Euphorbia supina extract was applied, and the content of the included active ingredient (ellagic acid) was maintained constant such that the stability of the multilamellar formulation was confirmed. By summarizing the above, it could be confirmed that the Euphorbia supina extract of the present invention has the potential as a new raw material for cosmetic compositions having excellent skin antioxidation and skin inflammation improvement effects.

Hereinafter, the present invention will be described in more detail in the following Examples and Experimental Examples. However, the scope of the present invention is not limited only to the following Examples and Experimental Examples, and includes all modifications of the technical idea equivalent thereto.

### Example 1: Preparation of Euphorbia supina Extract and Selection of Optimal Extraction Solvent

In this Example, an optimal extraction solvent for the Euphorbia supina extract was selected.

1) Preparation of Euphorbia supina Extract

In this Example, in order to select an extraction solvent, dried Euphorbia supina stems and leaves collected in the open field were extracted by applying a method of hot water extraction with distilled water, which was 10 times the amount of the sample, at 90° C. for 2 hours, and a method of extracting with 70% ethanol, which was 10 times the amount of the sample, at 50° C. for 2 hours. The Euphorbia supina extract which was extracted by each method was centrifuged at 4,000 rpm for 10 minutes, and the supernatant was used in the experiment after filtering with a 0.45 μm syringe filter and freeze drying.

2) Measurement of Antioxidant Efficacy of Euphorbia supina Extract-DPPH Scavenging Activity (%)

2,2-Diphenyl-1-picrylhydrazyl radical (DPPH) scavenging activity was applied to confirm the antioxidant efficacy of the extracts against free radicals, and the electron donating ability was measured by modifying the method of Kim et al (refer to Mathematical Equation 1). 0.2 mL of the sample was added to 0.8 mL of a 0.4 mM DPPH solution dissolved in ethanol, shaken for 5 seconds with a vortex mixer and left in a dark room for 10 minutes, and absorbance was measured at 517 nm.

$$Electron donating ability ( \% ) = \{ 1 - [ \frac{{A}_{Experiment} - {A}_{Blank}}{{A}_{Control}} ] \} \times 100 \qquad [ Mathematical Formula 1 ]$$

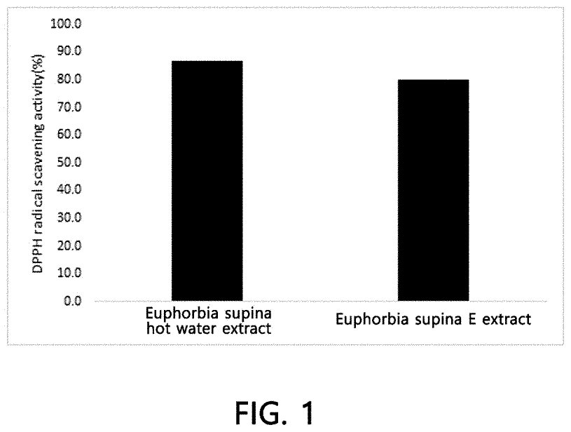

As a result, as shown in FIG. 1, the Euphorbia supina hot water extract showed a DPPH scavenging activity of 86.7% and the Euphorbia supina ethanol extract showed a DPPH scavenging activity of 79.8%, confirming that the hot water extract showed a superior DPPH scavenging activity.

3) Confirmation of Total Polyphenol and Flavonoid Contents of Euphorbia Supina Extract

For the total polyphenol content of the Euphorbia supina extract, 10 μL of the Folin-Ciocalteu phenol reagent and 100 μL of distilled water were added to 5 μL of the extract according to the Folin-Denis method, and then mixed at room temperature for 3 minutes. 100 μL of a 20% Na₂CO₃ solution was added to the mixture, and after reacting at room temperature for 1 hour, absorbance was measured at 725 nm using a spectrophotometer. Gallic acid was prepared in a concentration range of 25 to 800 μg/mL, and the total polyphenol content of the sample extract was calculated from the standard calibration curve obtained by analyzing in the same way as the sample.

For the total flavonoid content, 0.1 mL of 10% aluminum nitrate, 0.1 mL of 1 M potassium acetate and 4.3 mL of ethanol were sequentially added to 0.5 mL of the extract according to the method of Moreno et al, and after standing for 40 minutes at room temperature, absorbance was measured at 415 nm. By using quercetin (Sigma Co., USA) as a standard material, the total flavonoid content of the extract was calculated from the standard calibration curve obtained in a concentration range of 0 to 100 ug/mL.

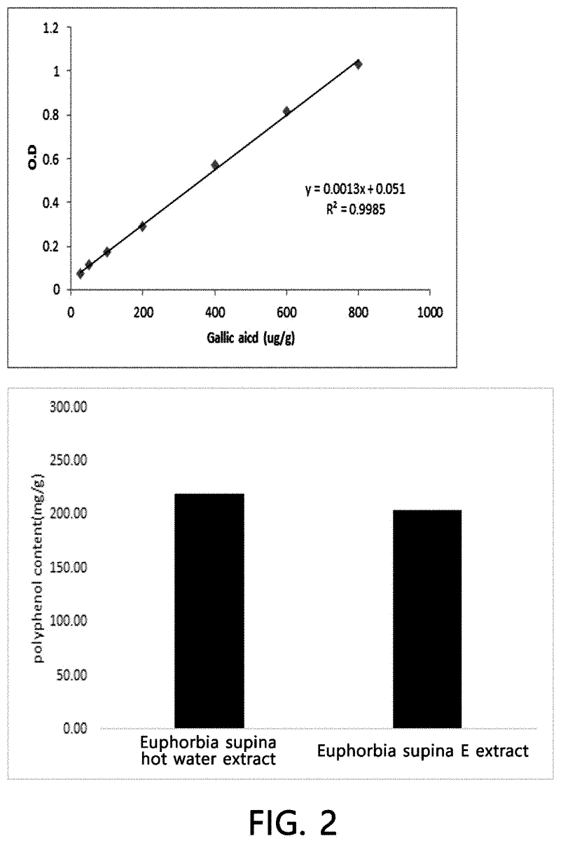

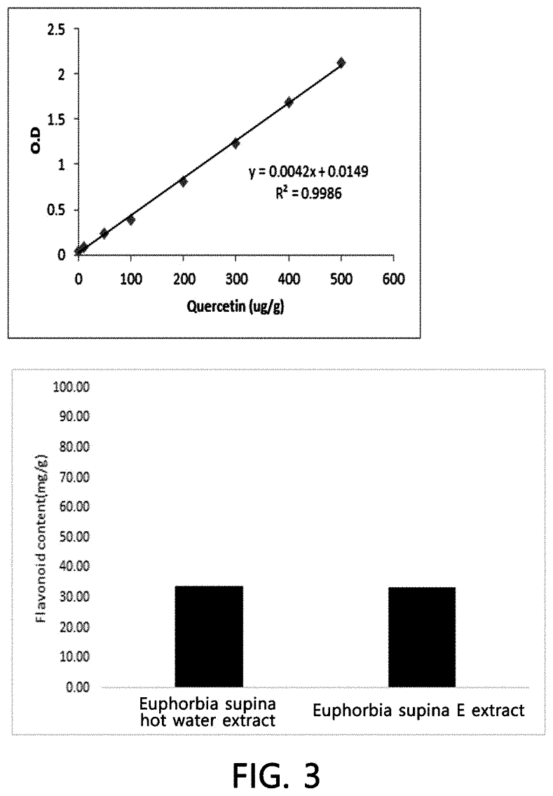

As a result, as shown in FIG. 2, the Euphorbia supina hot water extract showed a higher polyphenol content at 220 mg/g than the Euphorbia supina ethanol extract at 203 mg/g. In addition, as shown in FIG. 3, in terms of the flavonoid content, it was found that the Euphorbia supina hot water extract showed a similar content at 33.8 mg/g compared to the Euphorbia supina ethanol extract at 33.3 mg/g

4) Analysis of Marker Compounds in Euphorbia supina Extract

4-1) Selection of Marker Compounds

Hallym University provided 10 types of major compounds [gallic acid, quinic acid, ethyl gallate, gallic acid glucoside, ellagic acid, quetcetin 3-pentoside, quetcetin 3-pentoside, quercetin 3-hexoside, 1,6-digalloyl glucose, HHDP galloyl hexoside and heterophylliin A] obtained as a result of component analysis of Euphorbia supina through LC_MS.

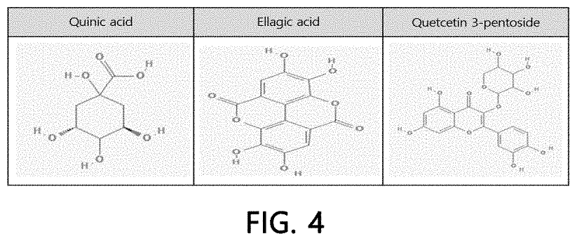

Data investigation was conducted in various ways to confirm the suitability of 10 types of the compounds as cosmetic materials, such as cosmetic effects and the like, and as a result, three types of quinic acid, ellagic acid and quetcetin 3-pentoside were selected as marker compounds of the Euphorbia supina extract (Table 1 and FIG. 4), and among these, the content analysis was performed for each extraction solvent of Euphorbia supina for ellagic acid through HPLC analysis, and it was selected as the final marker compound and confirmed by cross-validation with accredited institutions.

TABLE 1
T hree types of selected marker compounds

| | | m/z | Fragmentation | Major Cosmetic |
|---|---|---|---|---|
| Compound | Formula | [M − H]− | (m/z) | Function |
| Quinic acid | C₇H₁₂O₄ | 191 | 284, 257, 229, 185 | Anti-oxidant |
| Ellagic acid | C₁₄H₆O₈ | 301 | 301, 284, 229, 145 | Anti-oxidant, |
| | | | | anti- |
| | | | | inflammation |
| Quetcetin | C₂₀H₁₈O₁₁ | 433 | 433, 300, 283, 271, | Anti-oxidant |
| 3-pentoside | | | 255, 179, 151 | |

4-2) Analysis of ellagic acid content using HPLC

Ellagic acid of the Euphorbia supina extract was analyzed by using high performance liquid chromatography (HPLC) (Table 2 and Table 3). HPLC analysis conditions were separated using mobile phase A: 1% formic acid in DI and B: using acetonitrile under gradient conditions at a flow rate of 1 mL/min, and a Luna C18 (250×4.6 mm, 5 μm) column was used. The wavelength used for the detection of ellagic acid was 254 nm, and the concentration used to draw the calibration curve was 5 to 200 μg/mL.

TABLE 2
HPLC analysis conditions for quantification of ellagic acid

| Parameters | Conditions | |
|---|---|---|
| Standard | Ellagic acid | |
| Column | Luna C18 (250 × 4.6 mm, 5 μm) | |
| Column temp. | 40° C. | |
| Flow rate | 1 | mL/min |
| Injection volume | 10 | μL |
| Mobile phase | A: 1% formic acid in water (v/v), | |
| | B: 100% acetonitrile (v/v) | |
| UV wavelength | 254 | nm |

TABLE 3
Concentration according to time of mobile phase

| Gradient | Time (min) | A (%) | B (%) |
|---|---|---|---|
| | 0 | 75 | 25 |
| | 8 | 75 | 25 |
| | 10 | 10 | 90 |
| | 35 | 10 | 90 |
| | 37 | 75 | 25 |
| | 45 | 75 | 25 |

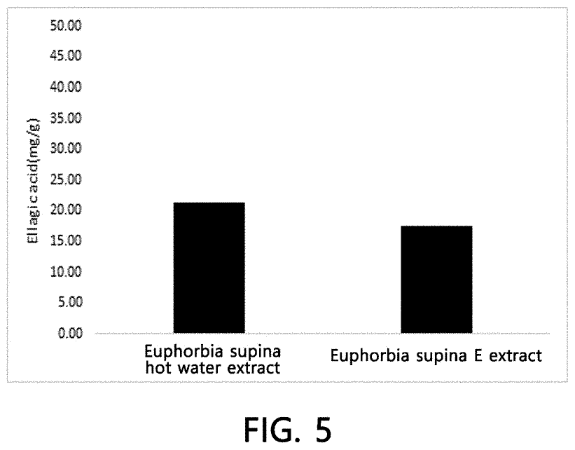

As a result, as shown in FIG. 5, the content of ellagic acid was measured to be 21.45 mg/g in the Euphorbia supina hot water extract and 17.49 mg/g in the Euphorbia supina ethanol extract, and it was found that the content in the Euphorbia supina hot water extract was 23% higher. Based on the DPPH scavenging activity, total polyphenol content, flavonoid content, ellagic acid analysis and the like, it was confirmed that hot water was the optimal extraction solvent for Euphorbia supina.

### Example 2: Selection of Optimal Temperature and Time for Euphorbia Supina Hot Water Extract of the Present Invention

In this example, the Euphorbia supina hot water extract was obtained using hot water which was confirmed as the optimal solvent above, and the optimal temperature and time were selected through various verifications.

1) Temperature and Time Settings

0° C., 30° C., 50° C., 70° C. and 90° C., and 2 hours and 4 hours were set.

2) Measurement of Antioxidant Efficacy of Euphorbia supina Hot Water Extract-DPPH Scavenging Activity (%)

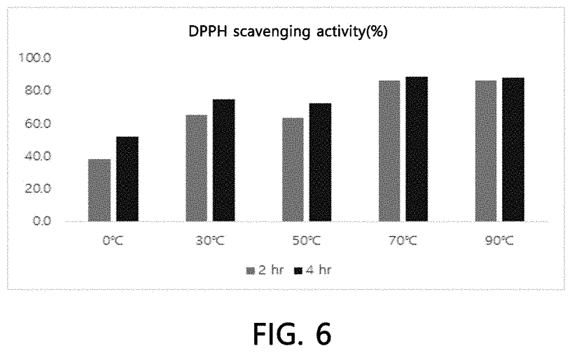

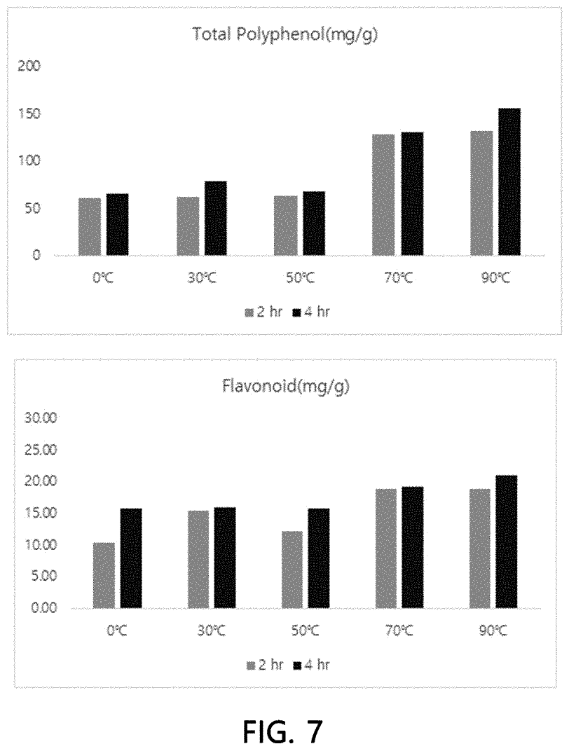

As used in the above Example, it was measured by modifying the method of Kim et al., and the results are shown in Table 4 and FIG. 6.

TABLE 4
Antioxidant efficacy results

| Extraction temperature | DPPH radical |
|---|---|
| and time | scavenging activity (%) |
| 0° C., 2 hr | 37.7 |
| 0° C., 4 hr | 51.9 |
| 30° C., 2 hr | 65.3 |
| 30° C., 4 hr | 74.7 |
| 50° C., 2 hr | 63.0 |
| 50° C., 4 hr | 72.5 |
| 70° C., 2 hr | 86.0 |
| 70° C., 4 hr | 88.8 |
| 90° C., 2 hr | 86.1 |
| 90° C., 4 hr | 87.9 |

As a result, it was confirmed that as the extraction time was longer and the extraction temperature was higher, the DPPH scavenging activity was higher.

3) Confirmation of Total Polyphenol and Flavonoid Contents of Euphorbia Supina Hot Water Extract

As used in the above Example, the total polyphenol content was measured according to the Folin-Denis method, and the total flavonoid content was measured according to the method of Moreno et al., and the results are shown in Table 5 and Table 7.

TABLE 5
Total polyphenol and flavonoid content results

| Extraction temperature | Total polyphenol | Total flavonoids |
|---|---|---|
| and time | content (mg/g) | content (mg/g) |
| 0° C., 2 hr | 60.41 | 10.39 |
| 0° C., 4 hr | 64.97 | 15.65 |
| 30° C., 2 hr | 61.63 | 15.44 |
| 30° C., 4 hr | 78.30 | 15.95 |
| 50° C., 2 hr | 26.30 | 12.11 |
| 50° C., 4 hr | 67.97 | 15.75 |
| 70° C., 2 hr | 127.63 | 19.08 |
| 70° C., 4 hr | 129.97 | 18.78 |
| 90° C., 2 hr | 131.97 | 18.78 |
| 90° C., 4 hr | 155.97 | 20.90 |

As a result, the polyphenol content showed the highest yield at 155 mg/g when extracted at 90° C. for 4 hours, and the flavonoid content showed the highest yield at 20.90 mg/g when extracted at 90° C. for 4 hours. Accordingly, it was confirmed that as the extraction time was longer and the extraction temperature was higher, the polyphenol and flavonoid contents increased.

4) Analysis of Ellagic Acid Content of Euphorbia supina Hot Water Extract

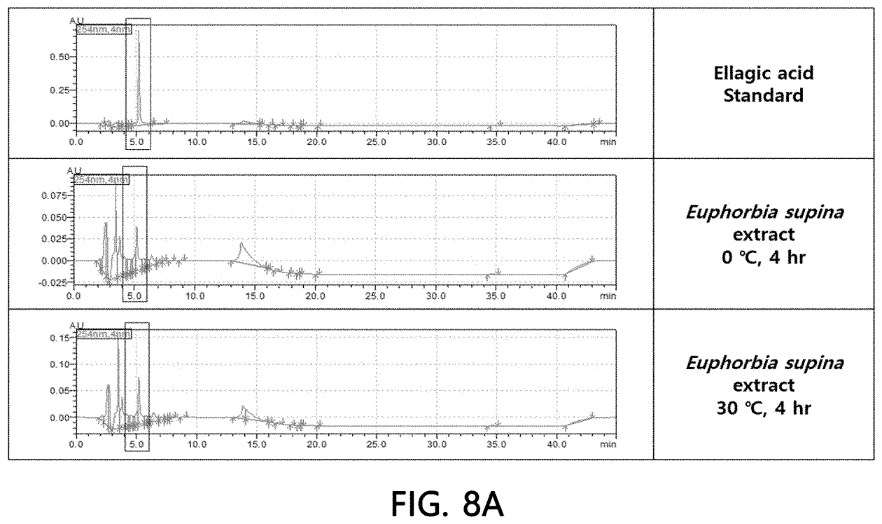

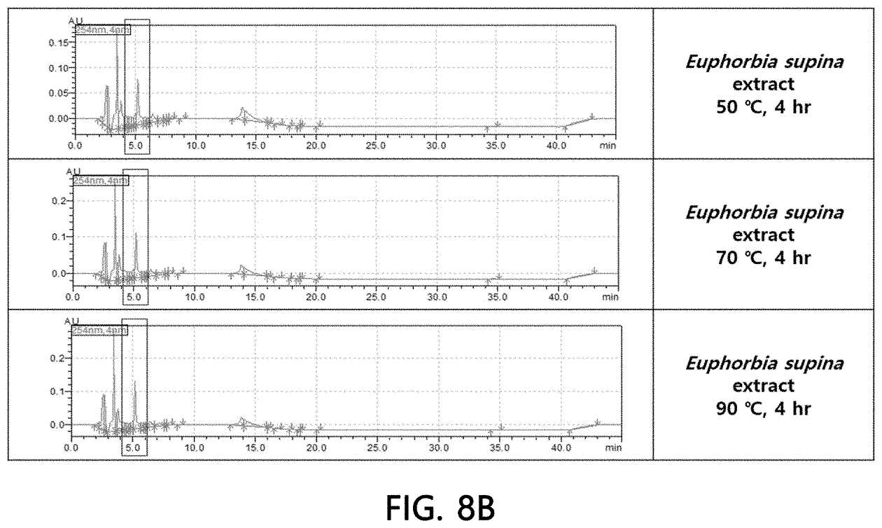

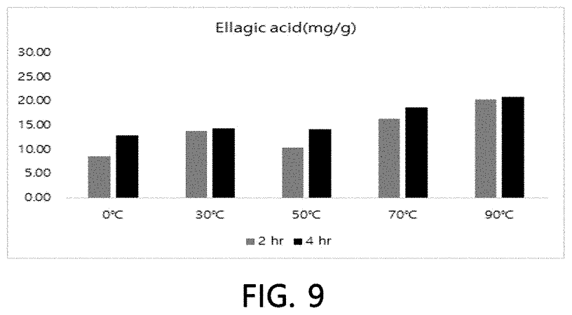

As used in the above Example, the ellagic acid content was analyzed by HPLC, and the results are shown in Table 6 and FIG. 8A, FIG. 8B and FIG. 9.

TABLE 6
Ellagic acid content results

| Extraction temperature | Ellagic acid |
|---|---|
| and time | content (mg/g) |
| 0° C., 2 hr | 8.59 |
| 0° C., 4 hr | 12.79 |
| 30° C., 2 hr | 13.76 |
| 30° C., 4 hr | 14.22 |
| 50° C., 2 hr | 10.32 |
| 50° C., 4 hr | 14.04 |
| 70° C., 2 hr | 16.20 |
| 70° C., 4 hr | 18.62 |
| 90° C., 2 hr | 20.21 |
| 90° C., 4 hr | 20.83 |

As a result, the ellagic acid content was 20.21 mg/g when extracted at 90° C. for 2 hours, and 20.83 mg/g when extracted for 4 hours, showing a higher yield than other temperatures and time periods, and it was confirmed to be identical to the HPLC results above.

5) Comparison of Changes in Ellagic Acid Content and Antioxidant Activity According to Hot Water Extract Conditions of Euphorbia supina

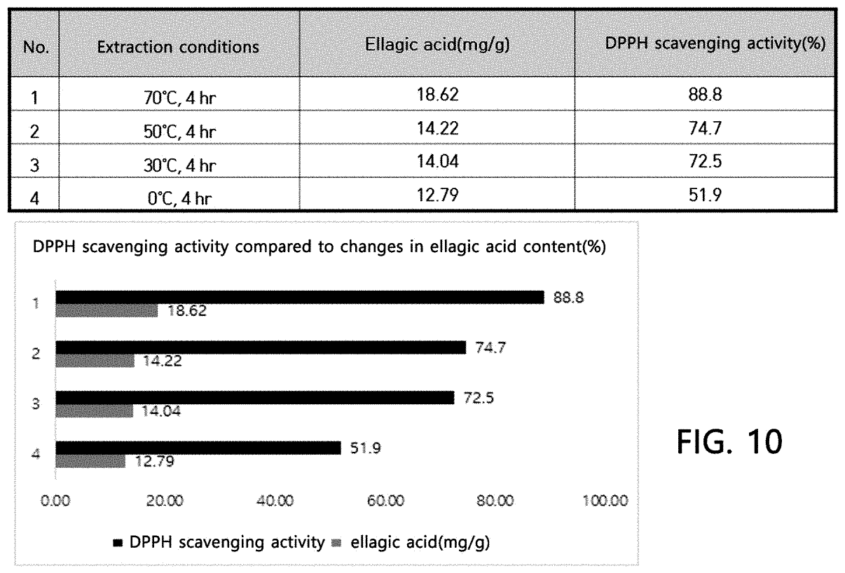

Changes in the ellagic acid content and DPPH scavenging activity measured by the hot water extraction conditions of Euphorbia supina were observed, and as shown in FIG. 10, the ellagic acid content was the highest during extraction at 70° C. for 4 hours, and it was confirmed that the DPPH scavenging activity also increased significantly in proportion.

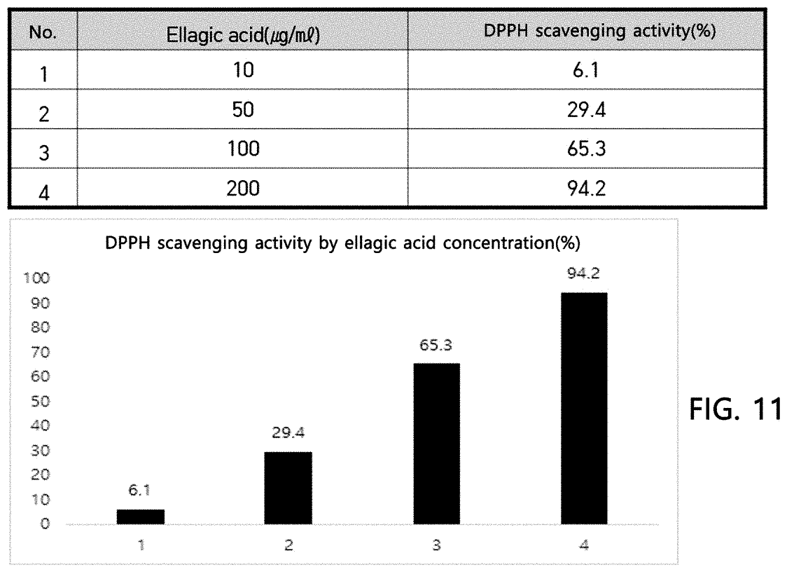

These results show that not only does ellagic acid exhibit various cosmetic effects, but also these were results confirming that it was selected as a representative marker compound, because it was judged to be a component for which QC was possible when considering that the DPPH scavenging activity of the crude extract (FIG. 10) also similarly increased compared to the DPPH scavenging activity of the ellagic acid content in a standard product (FIG. 11).

### Experimental Example 2: Validation of Analysis Method of Active Ingredient of Euphorbia supina Hot Water Extract

1) HPLC Analysis

It was carried out under the same conditions as the ellagic acid content analysis in Table 2 and Table 3 above.

2) Validation for Effectiveness of HPLC Analysis Method (Method Validation)

Validation for the effectiveness of the analytical methods for marker compounds was performed according to the validation guidelines for the analytical methods of pharmaceutical products and the like (KFDA, 2008) by evaluating linearity, the limit of detection (LOD), the limit of quantitation (LOQ), accuracy and the like. Ellagic acid, which is one of the active ingredients of Euphorbia supina, was proposed as a marker compound and the validation of the analysis method thereof was evaluated. The conditions of HPLC using Luna C18 (250×4.6 mm, 5 μm) with a detection wavelength of 254 nm were applied such that the analyte may be separated without interference from other substances through specificity testing (Table 2 and Table 3). A positive result obtained by using a sample including the analyte and a negative result obtained by using a sample consisting only of a matrix that did not contain the analyte were compared with the result obtained from a known standard product of the analyte.

2-1) Specificity

Specificity is the selective and accurate measurement of the analyte in the presence of impurities, degradation products, additives and the like, and if there is specificity in the used test method, it means that the detected signal is derived only from the analyte component and is not disturbed by the signals of other coexisting components, and thus, specificity refers to the discriminating ability of a test method and is also called selectivity.

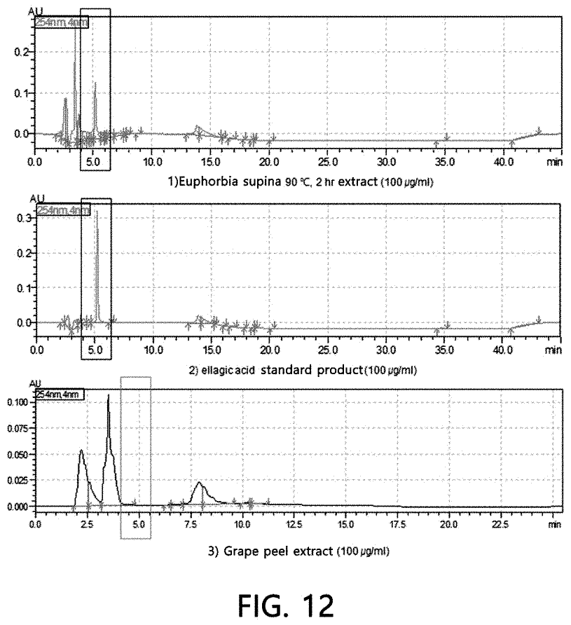

As a result of analyzing the Euphorbia supina extract by HPLC (FIG. 12), it was confirmed that the peak detected at 5 min of Euphorbia supina was the same substance as compared with the peak detected at 5 min of the standard product of ellagic acid. On the other hand, the same peak could not be detected within the same time in the grape peel extract, which was expected not to contain ellagic acid. Accordingly, it was possible to verify the specificity of ellagic acid in the Euphorbia supina extract.

2-2) Linearity

Linearity means that the test method may obtain a linear measurement value with respect to the amount (or concentration) of the analyte in the sample within a certain range, and it visually evaluates the linearity of a plot as a function of the concentration or content of the analyte. As a result of analyzing the Euphorbia supina extract under the established analysis conditions, it was confirmed that it was possible to analyze without interference even in a mixed state of the extract and showed the same spectrum in the UV spectrum of the ellagic acid standard solution and the Euphorbia supina extract, and verification of the analysis method was performed as follows.

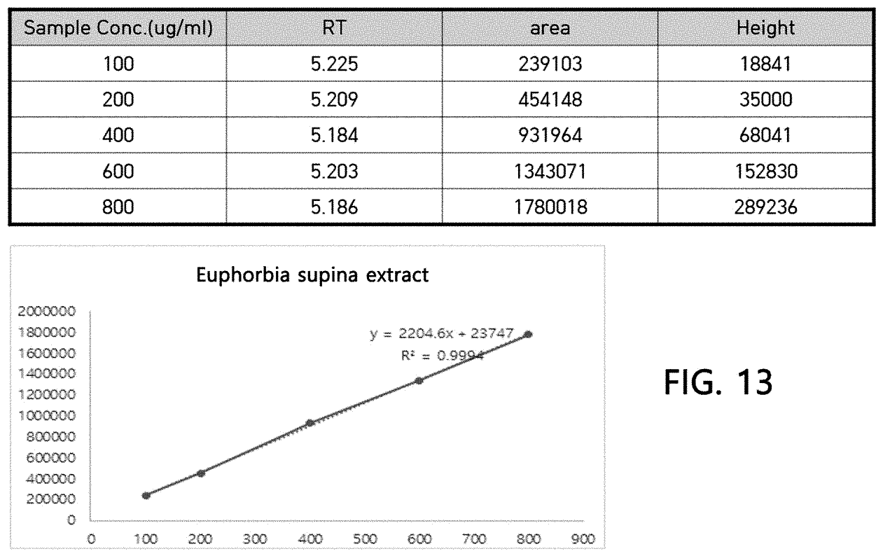

The standard solutions of ellagic acid were prepared to have 5 concentrations and the experiment was conducted, and for the standard solutions, the determination coefficient (R2) was confirmed using the retention time (RT) and regression equation (y=ax+by: peak area, x: concentration (μg/mL)) on HPLC. In addition, the limit of detection and the limit of quantitation for each component were calculated based on the standard deviation and the slope of the calibration curve using the chromatogram of the standard solutions, and the value of the determination coefficient (R2) was 0.9991 to 0.9994, which was almost similar to 1, showing high linearity (FIG. 13).

2-3) Limit of Detection (LOD) and Limit of Quantitation (LOQ)

The limit of detection and the limit of quantitation for each component were calculated based on the standard deviation and the slope of the calibration curve using the chromatograms of the standard solutions, and as for the limit of detection of ellagic acid, the test method showed the analysis concentration for each component and the low limit of detection and limit of quantitation such that the limit of detection and limit of quantitation for the analysis of the marker compound were verified (Table 7).

TABLE 7
Linearity, LOD and LOQ for ellagic
acid of Euphorbia supina extract

| | Linear | Re- | Re- | | | |
|---|---|---|---|---|---|---|
| | range | sponse | sponse | | LOD | LOQ |
| Compounds | (μg/mL) | slope | factor | R² | (μg/mL) | (μg/mL) |
| Ellagic acid | 100 to 800 | 2204.6 | 23,747 | 0.9994 | 1.12 | 8.23 |

2-4) Accuracy

Three samples were analyzed and measured by repeating three times of mixing a standard product of ellagic acid at low and high concentrations, respectively, and the recovered amount was confirmed intra-day and inter-day. The accuracy of ellagic acid was confirmed to be within 100.0 to 101.0%, which satisfied the recovery rate error of 90 to 110%, which is the guideline of the Ministry of Food and Drug Safety.

TABLE 8
Accuracy of ellagic acid contained in Euphorbia supina extract

| | Spiked | Detected | Accuracy (%) | |
|---|---|---|---|---|
| | Conc. | Conc. | Recovery¹⁾ | Recovery¹⁾ |
| Compounds | (μg/mL) | (μg/ml) | (Intra-day²⁾) | (Inter-day³⁾) |
| Ellagic acid | 100 | 99.53 ± 0.51 | 100.0 | 100.0 |
| | 250 | 297.47 ± 0.37 | 100.2 | 99.5 |
| | 500 | 495.92 ± 0.73 | 101.3 | 100.1 |

¹⁾ Recovery (%): [(Amount found − Original amount)/Amount spiked] * 100
²⁾ Intra-day: three times per day
³⁾ Inter-day: one time analysis of ellagic acid per day for 3 days

2-5) Precision

Precision is a coefficient variation (c.v.), and ellagic acid showed a decent value of 3.106%, which satisfied the relative standard deviation of 5% or less, which is the guideline standard value of the Ministry of Food and Drug Safety.

TABLE 9
Precision of ellagic acid contained in Euphorbia supina extract

| | Sample | | Amount | Average | SD | RSD¹⁾ |
|---|---|---|---|---|---|---|
| Compounds | (μg/mL) | Area | (μg/g) | (μg/g) | (μg/g) | (%) |
| Ellagic acid | 541.76 | 802.32 | 4.575 | 4.709 | 0.1462 | 3.106 |
| | 543.12 | 853.30 | 4.632 | | | |
| | 544.48 | 903.99 | 4.698 | | | |
| | 547.20 | 985.32 | 4.685 | | | |
| | 549.92 | 1063.23 | 4.956 | | | |

¹⁾ RSD: relative standard deviation

### Example 3: Development of Multilamellar Formulation Containing Euphorbia supina Extract

In this Example, a multilamellar formulation containing the Euphorbia supina extract was developed.

1) Phospholipid Used to Prepare Liposomes

The phospholipid used in the preparation of liposome was soy lecithin, which is lecithin in which lipids extracted from soybean were hydrolyzed to remove unsaturated components, and Emulmetik 950 (Lucas Meyer) with a phosphatidylcholine (PC) content of 95% or more in lecithin was used, and when the PC content was high and the hydrophile-lipophile balance (HLB) value was about 8, the surface activity was excellent and the high emulsifying power was exhibited.

It was prepared by mixing a 20% Euphorbia supina extract (the hot water and ethanol extracts) with a liposome base raw material, and hydrogenated lecithin was used as the emulsifier, and the raw materials required for liposome preparation were measured and used for each phase (aqueous/oily phases) according to the prescription.

2) Preparation of Multilamellar Liposome

It was prepared by modifying based on the Bangham method, and the steps are as follows.
Step 1: Phospholipids (Emulmetik 950+cholesterol) were dissolved in propanediol (the concentration of the phospholipids was adjusted to be less than 5%, and it was composed with cholesterol at less than 1%).
Step 2: A lipid membrane was formed by drying with a vacuum concentrator.
Step 3: After making the Euphorbia supina extract (the hot water and ethanol extracts) to 2% or less of the solid content on the lipid membrane, it was hydrated with an aqueous solution.
Step 4: For homogenization of the hydrated liposome solution, a homogenizing or ultrasonic homogenizer was used.

3) Confirmation of Multilamellar Liposome Formulation

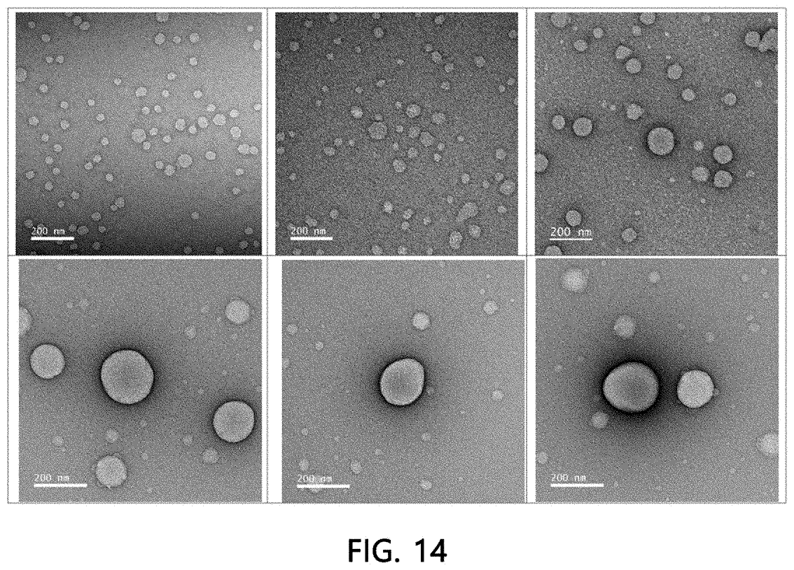

The size and shape of particles in the liposome formulation were observed with a field emission transmission electron microscope (FE-TEM), and spherical particles having a size of several tens of nanometers in the completed liposome formulation were confirmed by FE-TEM (FIG. 14).

### Experimental Example 3: Evaluation of Skin Permeability of Multilamellar Liposome Formulation Prepared in Example 3

In this Experimental Example, the skin permeability of the liposome formulation prepared in Example 3 was compared to that of the general formulation. To this end, analysis was conducted using the SKIN PAMPA system, which is a product of PION.

First, a blank was prepared with a prisma buffer and analyzed (reading). Next, as a process of preparing a sample in a deep well plate, 1,000 μL of the prisma buffer was added to the deep well plate according to the pH-map, the lid of the stock plate was opened, and 5 μL of the sample was transferred to the deep well plate using an 8-channel pipettor. Then, after the pipette was adjusted to 500 μt, it was mixed 4 to 6 times, and the concentration of the solution was measured or calculated, and the result was used as a <reference> of the assay.

Next, as a process of preparing a donor plate for the skin test assay, 200 μt of the sample solution from the deep well plate was transferred to a donor plate, and the lid was covered. In this case, the top plate of the PAMPA sandwich was an acceptor plate, and the bottom plate of the PAMPA sandwich was a donor plate.

Next, as a process of preparing a PAMPA sandwich and an acceptor plate for the skin test assay, after hydrating the well and preparing the assay of the donor plate, the top plate was transferred from the hydration solution reservoir to the support plate. Then, 200 μt of the prisma buffer at pH 7.4 was added to each well of the acceptor plate, and after removing the lid of the donor plate, it was slowly placed on the donor place from the bottom row (H) of the acceptor plate, while taking care not to create bubbles. In this case, after the sandwich was made, the lid was placed on the acceptor plate, and the experiment should be started within 4 to 5 minutes to prevent the skin membrane from drying out. Then, the sandwich was placed in a humidity chamber or gut-box with a wet sponge to maintain high relative humidity to minimize evaporation. Then, it was incubated without stirring for 5 hours at RT.

Next, as a process of analyzing the donor/acceptor (reading), after incubation was completed, the PAMPA sandwich was taken out from the humidity chamber, the lid was opened, and after transferring 150 μt from the acceptor plate to the UV plate using an 8-channel pipettor, it was set in a range of 250 to 498 nm in a spectrophotometer and analyzed (reading).

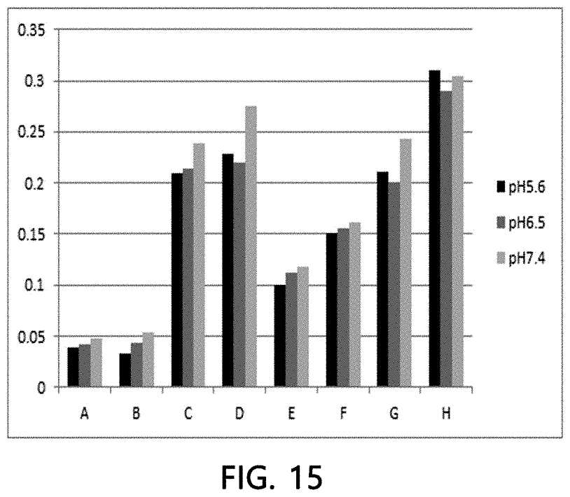

The results were as shown in FIG. 15, and it was confirmed that the skin permeability was significantly increased in the Euphorbia supina extracts (G, H) in the liposome formulation than in the Euphorbia supina extracts (E, F) that were not treated with the liposome formulation. In addition, although the skin permeabilities of the Euphorbia supina hot water extract and ethanol extract, which were not treated with the liposome formulation, were lower than those of the positive control groups (C, D), it was confirmed that it was significantly increased compared to the negative control groups (A, B). In addition, regardless of the presence or absence of the liposome formulation, it was confirmed that the Euphorbia supina hot water extract had a relatively high skin permeability compared to the ethanol extract. In general, normal skin exhibits weak acidity (pH 5.2 to 6.5), and acne or atopic skin is slightly alkaline (pH 7.5 to 8.0), and thus, cosmetics (creams, essences, toners, cleansers, etc.), to which the liposome formulation containing Euphorbia supina is applied, are expected to show excellent efficacy in skin whitening, wrinkle improvement and the like.

### Experimental Example 4: Stability Analysis of Multilamellar Liposome Formulation Prepared in Example 3

In this Experimental Example, the stability of the multilamellar liposome formulation was analyzed.

1) Formulation Stability: Acceleration Test

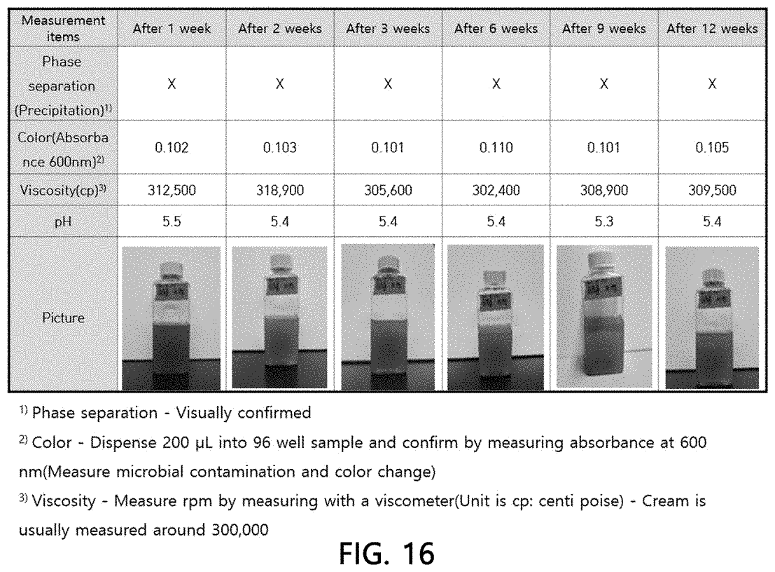

In order to confirm the stability of the liposome formulation in which the aqueous ellagic acid solution was collected, the stability for the phase separation, viscosity, color and odor of the formulation was evaluated for 12 weeks through acceleration experiments under the conditions of temperature 40±2° C. and relative humidity of 75±5% based on the guidelines of the Ministry of Food and Drug Safety, and the stability of the liposome formulation was confirmed (FIG. 16).

2) Stability of Materials (Raw Materials) in Formulation

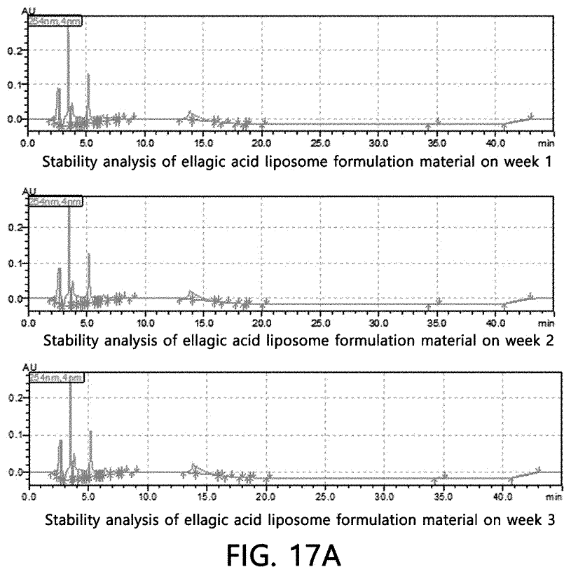

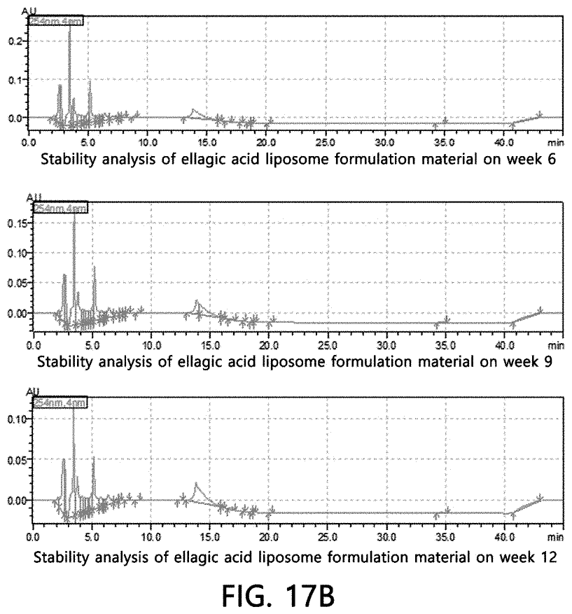

During the formulation stability experiment, samples were collected weekly and the stability of the raw materials in the product was analyzed by HPLC, and the results are shown in Table 10 and FIG. 17A and FIG. 17B.

TABLE 10
Analysis results of ellagic acid collection
rate in liposome formulation by period

| | Measurement period | | | | | |
|---|---|---|---|---|---|---|
| | Week 1 | Week 2 | Week 3 | Week 6 | Week 9 | Week 12 |
| Ellagic acid | 13.89 | 13.50 | 13.40 | 13.20 | 12.90 | 12.60 |
| content | | | | | | |
| (μg/mL) | | | | | | |

The first measured value of the ellagic acid content after liposome preparation, which was received as an official report, was 13.255 (μg/mL), and if the ellagic acid content measured periodically based on this value was 90% or more, it was evaluated as stable, and it was confirmed that the ellagic acid content was stable until the 12^{th} week.

### Example 4: Preparation of Cosmetic Composition (Cleanser) Using the Euphorbia supina Hot Water Extract of the Present Invention

In this Example, a cosmetic composition in a cleanser formulation using the Euphorbia supina hot water extract of the present invention was prepared. The detailed composition is shown in Table 11.

After weighing and completely dissolving phase B with an azimuth mixer for 20 minutes, the raw materials of phase A were weighed in phase B and heated at 80° C. for 1 hour to completely dissolve, and afterwards, it was cooled to room temperature, and the raw materials of phase C were added and dissolved slowly.

TABLE 11
Composition of cleanser formulation of Euphorbia supina hot water extract

| Phase | Name of raw materials | Content (%) | | |
|---|---|---|---|---|
| A | Purified water | 27.30 | 37.30 | 27.30 |
| | Lauryl glucoside | 10.00 | 5.00 | 10.00 |
| | Coco glucoside | 20.00 | 10.00 | 20.00 |
| | Decyl glucoside | 10.00 | 15.00 | 10.00 |
| | Sodium cocoamphoacetate | 10.00 | 10.00 | 10.00 |
| | Glycerin | 2.00 | 2.00 | 2.00 |
| | Honey | 1.00 | 1.00 | 1.00 |
| | Calendula flower extract | 1.00 | 1.00 | 1.00 |
| | Glyceryl caprylate | 0.50 | 0.50 | 0.50 |
| | Glycerin | 3.00 | 3.00 | 3.00 |
| | Butylene glycol | 5.00 | 5.00 | 5.00 |
| B | Xanthan gum | 0.20 | 0.20 | 0.20 |
| | Guar hydroxypropyl- | 0.15 | 0.15 | 0.15 |
| | trimonium | | | |
| | chloride | | | |
| | LMW | 0.05 | 0.05 | 0.05 |
| | Hyaluronic acid | 0.20 | 0.20 | 0.20 |
| | Purified water | 60.09 | 60.09 | 61.09 |
| | Aloe vera gel powder | 0.10 | 0.10 | 0.10 |
| | D-Panthenol | 0.50 | 0.50 | 0.50 |
| C | Euphorbia supina hot | 1.00 | 1.00 | 1.00 |
| | water extract | | | |
| | 1,2-Hexanediol | 1.00 | 1.00 | 1.00 |
| | Ethylhexylglycerin | 0.02 | 0.02 | 0.02 |
| | Citric acid | 0.04 | 0.04 | 0.04 |

| | | | |
|---|---|---|---|
| Total | 100.00 | 100.00 | 100.00 |
| | Example 4-1 | Example 4-2 | Example 4-3 |

As a result of the usability test for the cosmetics in the formulation prepared above, the product with the best foaming power and soft touch was Example 4-2, and the removal of dead skin cells and moisturizing power were the most excellent.

1. A cosmetic composition for antioxidation, comprising a Euphorbia supina extract.

2. The cosmetic composition of claim 1, wherein the Euphorbia supina extract is extracted with 60% to 80% ethanol or hot water.

3. The cosmetic composition of claim 2, wherein the Euphorbia supina extract is collected in an aqueous layer of multilamellar liposome.

4. A cosmetic composition for improving skin inflammation, comprising a Euphorbia supina extract.

5. The cosmetic composition of claim 4, wherein the Euphorbia supina extract is extracted with 60% to 80% ethanol or hot water.

6. The cosmetic composition of claim 5, wherein the Euphorbia supina extract is collected in an aqueous layer of multilamellar liposome.

7. A method of skin care, comprising applying a Euphorbia supina extract to a skin.

8. The method of claim 7, wherein the Euphorbia supina extract is extracted with 60% to 80% ethanol or hot water.

9. The method of claim 8, wherein the Euphorbia supina extract is collected in an aqueous layer of multilamellar liposome.

10. The method of claim 7, wherein the method is for skin antioxidation or improving skin inflammation.
